(19) United States
(12) Plant Patent Application Publication
(10) Pub. No.: US 2003/0056268 P1
(43) Pub. Date: Mar. 20, 2003

(54) Chrysanthemum plant named 'Prize Time'

(76) Inventor: Mark Roland Boeder, J H Den Haag (NL)

Correspondence Address: HOWREY SIMON ARNOLD & WHITE, LLP, BOX 34, 301 RAVENSWOOD AVE., MENLO PARK, CA, 94025, US

(21) Appl. No.: 09/957,491

(22) Filed: Sep. 19, 2001

(51) Int. Cl. A01H 5/00

(52) U.S. Cl. PLT/295

(57) ABSTRACT

A new variety of Chrysanthemum plant named ‘PRIZE TIME,’ having yellow single type flowers, a slightly domed capitulum form and an upright growth habit.

### BACKGROUND OF THE INVENTION

The present invention comprises a new and distinct cultivar of Chrysanthemum botanically known as Dendranthema grandiflora, and referred to by the cultivar name ‘Prize Time’.

‘Prize Time’, identified as 20223-0 originated from a cross made by Mark to Boeder, in a controlled breeding program in Chichester, West Sussex, United Kingdom.

The female parent of the new variety is an undistributed, unpatented seedling identified as P343B1 and the male parent is the variety known as P341B7. The new variety ‘Prize Time’ has been asexually reproduced by vegetative cuttings at Chichester, West Sussex, United Kingdom and the distinguishing characteristics are retained through successive generations of asexual reproduction.

### BRIEF SUMMARY OF THE INVENTION

‘Prize Time’ is a pot type of Chrysanthemum plant variety having a flat capitulum form and bright vibrant yellow flowers.

### Comparison with Parent

Plants of the new Chrysanthemum variety ‘Prize Time’ are similar to the parent cultivar P341B7 in plant habitat and growth rate. In side-by-side comparisons in Chichester, West Sussex, United Kingdom, under commercial practice, plants of the new Chrysanthemum variety ‘Prize Time’ differed from plants of the parent cultivar P341B7 in the following characteristics.

1. The new variety ‘Prize Time’ produces yellow single flowers whereas the parent cultivar P341B7 produces red single flowers.

2. Plants of the new variety ‘Prize Time’ have smaller and taller inflorescence than plants of the parent cultivar P341B7.

### Comparison with Other Varieties

Plants of the new Chrysanthemum variety ‘Prize Time’ are dissimilar to the cultivar ‘Mirimar’ in plant habitat and growth rate. However, in side-by-side comparisons in Chichester, West Sussex, United Kingdom, under commercial practice, plants of the new Chrysanthemum variety ‘Prize Time’ differed from plants of the cultivar ‘Mirimar’ in the following characteristics.

1. The new variety ‘Prize Time’ produces darker yellow single flowers, whereas the cultivar ‘Mirimar’ produces yellow single flowers.

2. Plants of the new variety ‘Prize Time’ have larger and taller inflorescence than plants of the cultivar ‘Mirimar.’

3. The new variety ‘Prize Time’ has a faster response time than the variety ‘Mirimar.’

### BRIEF DESCRIPTION OF ILLUSTRATIONS

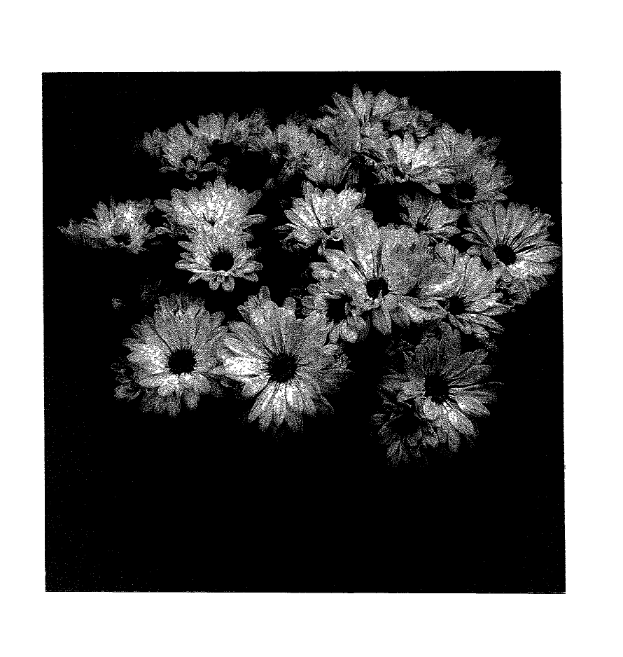

Typical specimens of the plant and flowers for the new Chrysanthemum variety ‘Prize Time’ are shown in the accompanying photograph. The colours shown are as true as possible within the usual limits of this kind of illustration. FIG. 1 is a whole plant view of the new variety ‘Prize Time’ grown in a pot. The plant shown in the illustration is 56 days from the start of Short Days.

### DETAILED BOTANICAL DESCRIPTION

The following description of the new Chrysanthemum variety ‘Prize Time’ is of plants grown in a greenhouse in Chichester, West Sussex, United Kingdom in the month of June. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 18° C. at night and about 24° C. to 25° C. during the day under light conditions of about 5,000 to 6,000 foot candles. The plants respond well to the use of growth retardant, such as three B9 treatments at about 2500 ppm. To produce a commercial product the plants may be pinched once with the centre bud removed. The typical container size for commercial growth is 14 cm. It has been observed that the shelf life of the new variety is about 24 days with a response time of about 7½weeks. The new variety is suitable for growth in a temperature range of 16° C. to 25° C.

The new variety may be produced as a spray or disbud. The following description is with respect to a plant produced as Spray pot Chrysanthemums. In the description of this new Chrysanthemum variety, colour values have been taken from the Royal Horticultural Society Colour Chart (R.H.S.C.C.).

### Plant

Plant type: Pot

Habit: Upright and branching

Height: Medium/Tall 20 to 23 cm.

Branching characteristics: Free and spreading.

Length of Lateral Branches: 12 to 17 cm.

Number of Breaks from Pinch: 5 to 7.

Stem Colour: 138 B.

Response Time: 52 days.

Vigour: Medium/Tall.

Shelf Life: Near 24 days.

Disease Resistance: No observations.

Pest Resistance: No observations.

Growth retardant type and treatment: B9 85% 3 applications of 2.5 gram/litre at 2 days, 21 days and 28 days after sticking of unrooted cuttings.

The plants were grown for two weeks in Long Day conditions (20 hours of light) and then transferred to Short Day conditions (13 hours of dark).

Propagation:

Type.—Vegetative propagation via stem cuttings

Time to rooting.—12-14 days with soil temperatures of 18° C.

Rooting habit.—Fine and fibrous

### Foliage

Number of Leaves per Lateral Branch: 6 to 10

Compound or single: Single

Arrangement of Leaves: Alternate

Shape of Leaf.—Typically 5 lobed, ovate

Size of Leaf.—Width (cm): Near 4½-6 cm Length (cm): Near 8-9½cm

Leaf Apex.—Acute

Base.—Obtuse

Attachment.—Petioled

Aspect.—Slightly undulating

Margin.—Palmately lobed

Surface characteristics.—Top: Slightly pubescent Bottom: Slightly pubescent

Petiole: Colour: Near 137 C

Length.—Near 2½cm

Venation: Net, prominent midvein and lobe veins prominent.

Colour.—upper side: Near 137 D under side: Near 137 D

Colour: Mature Leaf, upper side: Near 137 A; under side: Near 137 C Young Leaf, upper side: Near 137 A; under side: Near 137 D

### Flower

Flower type: Single (Daisy)

Flower shape: Circular, slightly cupped

Number of blossoms per branch: 3 to 5

Inflorescence form: Pyramidal

Depth of filly expanded blossoms.—Near 3½cm

Diameter of fully expanded blossoms.—Near 7 to 9 cm

Sepals:

Number.—21 to 25

Colour.—Near 137 B

Length.—0.5-1.2 cm

Width.—0.2-0.4 cm

Texture/Appearance.—Slightly pubescent

Pedicel:

Length.—Varies between 3 to 6 cm

Colour.—Near 138 B

Surface.—Pubescent

Habit.—Straight

Strength.—Medium

Ray Florets:

Form/Shape.—Between reflexing 1 and 2.

Texture/Appearance.—Matte.

Numberperflower.—41 to 50.

Length: 3 to 4½cm; Width: 0.5 to 1.0 cm.

Apex.—Round and dentate.

Base.—Tapered.

Margin.—Entire.

Disc florets:

Form/Shape.—Cylindrical.

Texture/Appearance.—Shiny.

Number perflower.—170 to 250.

Length.—Near 0.4 to 0.6 cm; Width: 0.1 to 0.1 cm.

Diameter of disc.—1.0 to 1½cm

Fragrance: Faint

Flower Bud (at onset of colour):

Length.—Near 0.7 cm.

Diameter.—Near 1.2 cm.

Form/shape.—Globular.

General flower colour:

1. Ray florets, upper side:

Immature.—Near 13 A.

Mature.—Near 13 B.

Older/Fading.—Near 13 C.

2. Ray florets, under side:

Immature.—Near 13 A.

Mature.—Near 13 B.

Older/Fading.—Near 134 C.

3. Disc florets:

Immature.—

Mature.—

Older/Fading.—

4. Bud: Near 137 B.

Flower progression: Flowers hold their colour during ageing.

### Reproductive Organs

Ray Florets: Pistillate

Stigma colour.—Near 6 C.

Stigma shape.—Forked.

Style Colour.—Near 6 C.

Style Length.—l Near 0.5 cm.

Disc Florets: Pistillate

Stigma colour.—Near 6 C.

Stigma shape.—Forked.

Style Colour.—Near 6 C.

Style Length.—Near 0.2 cm.

Staminate.—Not observed

What is claimed is:

1. A new and distinct variety of Chrysanthemum plant, substantially as described and illustrated herein.
